(19) United States
(12) Patent Application Publication
(10) Pub. No.: US 2007/0112357 A1
(43) Pub. Date: May 17, 2007

(54) Utilizing vibrational energy under 1000 hertz for dermatological infusion treatment modality

(76) Inventors: Jack Skinner, Grand Prairie, TX (US); Terry Osborn, Grand Prairie, TX (US)

Correspondence Address: Jenkens & Gilchrist;a Professional Corporation, Suite 3700, 1445 Ross Avenue, Dallas, TX, 75202, US

(21) Appl. No.: 11/472,089

(22) Filed: Jun. 21, 2006

Related U.S. Application Data
(60) Provisional application No. 60/692,443, filed on Jun. 21, 2005.

(51) Int. Cl. A61B 17/50

(52) U.S. Cl. 606/131

(57) ABSTRACT

A method of treatment for dermatological rejuvenation of a skin area. The method includes the utilization of vibratory motion of a surface applied to an individual's skin. The dermatological rejuvenation includes the abrasive removal of an outer layer of skin to reveal an underlying skin area, which is vibratorily infused with a cream or the like via a paddle.

### CROSS-REFERENCE TO RELATED APPLICATIONS

This application claims priority to and incorporates by reference the entirety of U.S. Provisional Application Ser. No. 60/692,443 filed Jun. 21, 2005.

### BACKGROUND OF THE INVENTION

1. Field of the Invention

The present invention relates to the use of vibrational energy under 1000 hertz for both dermatological and pain treatment modalities, and more particularly, but not by way of limitation, to a method of and apparatus for skin rejuvenation utilizing vibrational energy applied to a patient from a dermatological treatment system.

2. History of Related Art

A method of rejuvenating skin has been patented by Brian D. Zelickson in U.S. Pat. No. 6,645,184 (the '184 patent) wherein a selected tape is applied to the surface of an applicator tip adapted for select motion. One skin treatment product surface element therein described is “abrasive.” The inventors of the present invention, recognizing the usefulness of the abrasive tape surface of the system described in the '184 patent, attempted to design, and have made, a skin treatment paddle of substantially unitary assembly with a permanent abrasive surface for “removing excess skin cells.” The material from which certain sample paddles were made and tested was polypropylene. Other materials may be used. The surface pattern that was produced was determined to be not sufficiently abrasive for the desired skin treatment of “removing excess skin cells.” However, it was found by the inventors hereof that the surface pattern of the polypropylene paddle of unitary construction was ideal for “infusion” of substances into the skin. Further research confirmed this finding, and further development was undertaken. Additional testing has determined there are additional benefits and effects in the area of “pain management”.

Therapeutic devices offering pain relief through application of vibration are generally known and several are currently on the market today. Most of these devices utilize pads with large surface areas to massage the general areas of the head, neck, and back. One such prior art device, U.S. Pat. No. 5,314,456, entitled Therapeutic Pad For Relief Of Headache-Related Head, Temple, Neck, and Back Pain discloses a large rectangular pad with wings to wrap around a users head capable of providing therapeutic inputs. Another prior art device, U.S. Pat. No. 5,486,156, entitled Head Vibrator discloses a form-fitting skullcap that contains several vibration generators. Another prior art device, U.S. Pat. No. 6,554,787, entitled Headband For Treatment Of Headaches discloses a headband containing one or more pockets for the insertion of treatment elements including cold elements, heat elements, vibration elements, and magnetic elements. None of these devices has ability to apply pinpoint vibration to the body, especially the face.

Another class of therapeutic devices provide pain relief through muscle massaging. One such prior art device, U.S. Pat. No. 6,682,496 (the '496 patent), entitled Deep Muscle Stimulator Device discloses a handheld device with a rotating electrical motor that provides deep muscle tissue with kinetic forms of percussion and concussion vibration. The '496 patent additionally discloses that the device preferably operates at a speed of between 2000 and 3600 RPM to rotate a shaft and coupling system at that speed. The percussion and concussion motions of these devices is not suitable for use on the head or face region.

### BRIEF SUMMARY OF THE INVENTION

The present invention relates to a newly discovered effect of a dermatological system and method. More particularly, in one embodiment, a first effect of the invention is enhanced infusion of dermatological creams, lotions and the like. Still more particularly, in one embodiment of the invention, a vibratory paddle of the type having either a permanent, substantially abrasive surface or a substantially non-abrasive surface is placed on a facial skin area for infusion of lotions, creams or other substances. In another embodiment, a vibratory paddle having an abrasive surface adapted for removing an outer layer of skin surface and exposing an underlying skin area to subsequent substance infusion by a second vibratory paddle having a permanent, substantially non-abrasive surface adapted for oscillatory infusion at a rate of less than 1000 Hz is utilized to provide a dermatological effect.

In yet another embodiment of the invention, a vibrational skin rejuvenation system incorporating a plurality of devices having permanent abrasive surfaces adapted for selectively removing outer portions of a skin area and exposing an underlying skin portion for subsequent treatment is operated in the range of 80 to 100 Hz for lotion, cream or other substances infusion. In one embodiment, the device may be specially designed paddles. The system further includes a vibratory paddle having a permanent, substantially non-abrasive surface adapted for the kinetic infusion by vibratory action in the range of 80 to around 100 Hz of substances such as creams or lotions placed on the exposed skin area.

In yet another embodiment, the present invention includes a single paddle having permanent surfaces for the treatment of the skin in a vibratory oscillation mode less than 1000 Hz, and preferably in the range of 80 to 100 Hz for substance infusion. The paddle may be constructed with a first, permanent, abrasive side and an opposite, permanent, non-abrasive side adapted for kinetic infusion of substances into a skin area.

In yet another embodiment, one aspect of the invention includes using the abrasive surface or paddle described above to kinetically infuse substances into the skin of a patient after skin abrasion.

In yet a further aspect, one embodiment of the present invention includes a method for dermatological rejuvenation of a skin area of an individual utilizing vibratory motion of select dermatological treatment surfaces applied to the individual's skin. The method comprises the steps of providing a first dermatological treatment surface having a sufficiently abrasive side for removal of at least one outer skin area of an individual; removing said at least one outer skin area of said individual through abrasive vibratory treatment of at least one said skin area with said first paddle to expose an underlying skin area for the application of a cream, lotion or other dermatological aid to said exposed underlying skin area of said individual; and providing a second dermatological treatment surface having a substantially non-abrasive side containing a plurality of lands and grooves formed therein and infusing said cream, lotion, or other dermatological aid into said exposed underlying skin area of said individual through vibratory movement of said second surface at a frequency less than 1000 Hz.

In one embodiment, the method described above includes the step of positioning the lands and grooves of said second dermatological treatment surface over aid skin area to impart, by said vibratory movement, angular vectors to molecules of said cream, lotion, or other dermatological aid to drive said molecules into said exposed underlying skin area.

The method herein described may also include the step of providing a first paddle adapted to contain said first dermatological treatment surface upon one of said first paddle's sides. In one embodiment, the step of infusing utilizes vibratory movement of said dermatological treatment surface at a frequency on the order of the range of 80-100 Hz.

In another embodiment there is provided a system for dermatological rejuvenation of an individual's skin area. The system comprises a first dermatological treatment surface having a sufficiently abrasive side for vibratory removal of an outer skin area of an individual; a second dermatological treatment surface having a side specifically adapted for the vibratory infusion of a cream, lotion, or other dermatological aid into an underlying skin area of said individual; a wand adapted to transfer a vibrational energy to said first and second dermatological treatment surface; and a control unit adapted to connect to said wand and further capable of generating a vibrational energy in said wand at a frequency of less than 1000 Hz.

In yet another embodiment, the system described above includes the second dermatological treatment surface containing a plurality of lands of a regular shape defined by a pattern of recessed grooves, said lands being specifically adapted for vibratory infusion of said cream, lotion, or other dermatological aid at a frequency less than 1000 Hz, and in one embodiment at a frequency on the order of 80 to 100 Hz.

The above summary of the present invention is not intended to represent each embodiment or every aspect of the present invention.

### BRIEF DESCRIPTION OF THE DRAWINGS

A more complete understanding of the method and apparatus of the present invention may be obtained by reference to the following Detailed Description when taken in conjunction with the accompanying Drawings wherein:

FIG. 1 is a front elevational view of a dermatological vibrational unit adapted for the select vibratory treatment of a skin surface.

FIG. 2 is a front elevational view of an array of vibratory paddles adapted for use in conjunction with the present invention, one of which is shown to be mounted to a wand of the type shown in FIG. 1;

FIG. 3 is a diagrammatical illustration of the utilization of the wand of FIGS. 1 and 2 relative to a patient;

FIG. 4 is an enlarged front elevational view of a non-abrasive paddle constructed in accordance with the principles of the present invention and adapted for the infusion of substances into the skin of a patient;

FIG. 5 is an enlarged, diagrammatical, side elevational view of the vibratory action of the paddle of FIG. 4 relative to the skin area of a patient for illustrating the infusion angles imparted by the vibrational action of the paddle in accordance with one embodiment of the principles of the present invention;

FIG. 6 is a flow diagram of one embodiment of the dermatological aspects of the present invention;

FIG. 6A is a flow diagram;

FIG. 7 is a side elevational view of a two-sided paddle incorporating a permanent abrasive surface on a first side and a substantially non-abrasive surface on the second, opposite side; and

FIG. 8 is a diagrammatical illustration of one embodiment of the use of the vibrational unit of the present invention.

### DETAILED DESCRIPTION OF THE INVENTION

The vibrational unit 10 includes a control panel 12 on the frontal portion of a body 14. A wand 16 connected to the body 14 by a cord 18 facilitates the mounting of select dermatological (dermal) rejuvenation equipment, including vibratory paddles described below and in the above-referenced provisional patent applications incorporated herein in their entireties by reference. In one embodiment, a vibrational unit may be incorporated into a portable battery operated device.

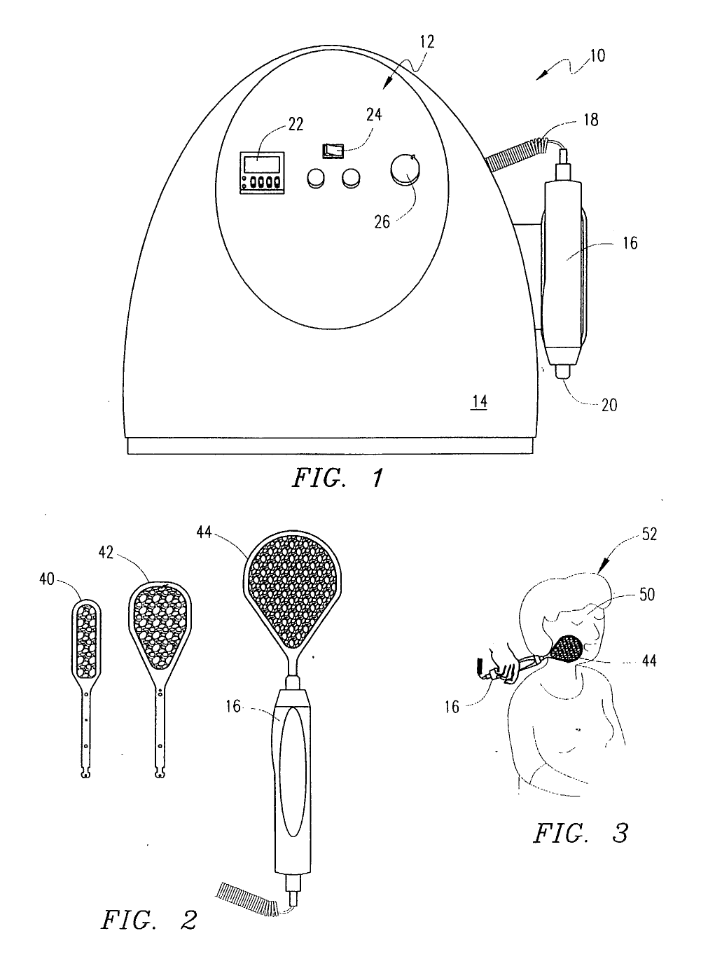

Still referring to FIG. 1, the wand 16 has a distal end 20 adapted for receipt of a plurality of paddles therein. Once the select paddle is inserted, the control panel 12 is utilized to actuate the vibrational energy for a select period of time. In that regard, a monitor 22 may be used relative to a power button 24 and a vibrational energy knob 26 for control of the kinetic energy being imparted to the skin of a patient.

Referring now to FIG. 2, there is shown an array of vibratory paddles adapted for dermatological rejuvenation. Paddle 40 is of elongate, unitary construction (that term is used herein to mean a “permanent” assembly with “no removable parts”) providing for vibrational rejuvenation in narrower face regions. Paddle 42 is likewise of wider construction allowing for more select vibrational dermatological applications. Paddle 44 shown mounted into wand 16 is similarly of a larger size adapted for a larger more readily available skin areas.

Referring now to FIG. 3, there is shown the utilization of the wand 16 and paddle 44 against the face and/or neck 50 of a patient 52. It may be seen that the vibrational energy imparted to the skin of the patient 52 imparts select energy thereto for the effective removal of outer skin cells from the face region 50. Other areas of the body are, of course, contemplated in accordance with the principles of exclusively dermatological uses of the unit 10. For headache pain, use of the wand 16 near the brain has been shown to provide headache relief. As presently know, use of the unit 10 in the range of 80 to 100 Hz has been shown to provide headache relief; even migraine headache relief.

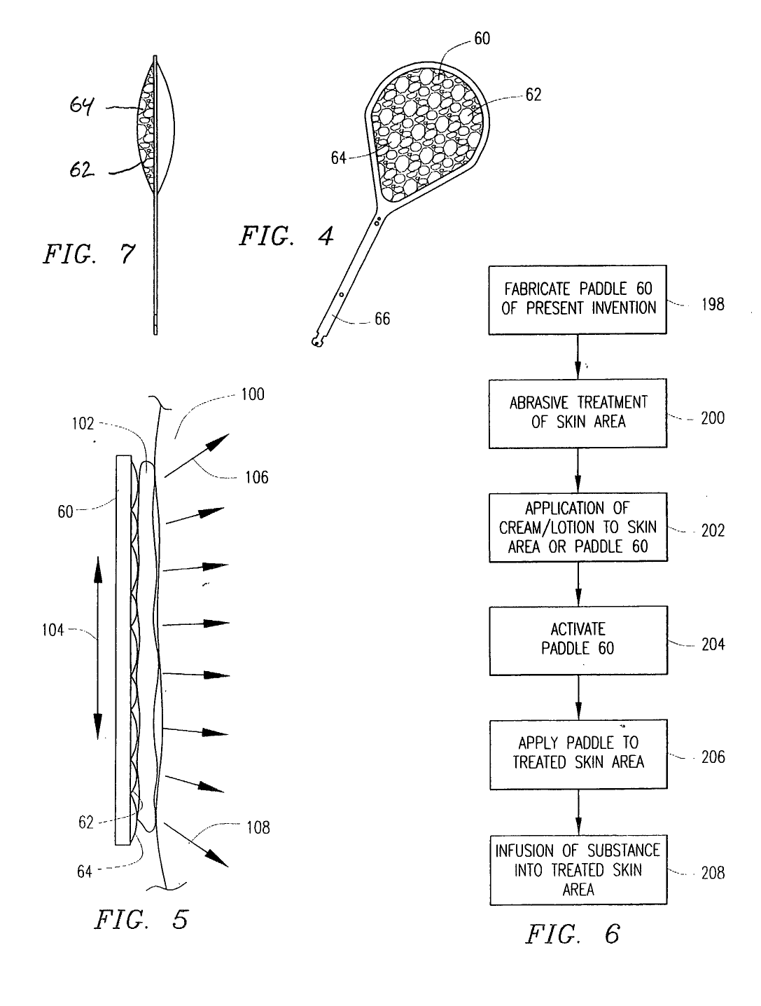

Referring now to FIG. 4, there is a shown a paddle 60 adapted for infusion of select substances into the skin of a patient. It may be seen that the paddle 60 of the present invention includes a frontal surface 62 having a defined pattern 64 thereon. The pattern 64 may vary depending on the particular applications, and the inventor hereof is in the process of evaluating an abrasive paddle designs may be used with effectiveness. It may be seen that a stem 66 of the infusion paddle 60 of the present invention is of similar construction to that of paddles 40 and 42 in FIG. 2, so that paddle 60 of the present invention may likewise be inserted into distal end 20 of wand 16 for application on a patient. The paddle 60 of the present invention may be fabricated from polycarbonate, polyethylene or polypropylene. The fabrication material has not, however, as of the date of this application, been shown to be material in the discovered pain treatment aspect. However, studies are underway.

Reference is again specifically made to the above-referenced provisional application, the entirety of which is incorporated by reference. In accordance with the principles of the present invention, a select substance such as a lotion or cream or other dermatological treatment ingredients are applied either to the face 50 or the paddle 60 of the present invention and/or both and vibrationally infused into the skin of patient 52. An abrasive paddle may likewise be used for infusion. When the substance infusion follows the dermatological abrasion represented in FIG. 3, the skin surface 50 of the patient 52 is generally in condition for more effective absorption of the select substance by means of the kinetic application set forth herein and further described in the aforesaid provisional applications.

Referring now to FIG. 5, there is shown an enlarged diagrammatical side-elevational view of the paddle 60 of the present invention against the skin area 100 of a patient. It may be seen that a lotion or cream 102 is illustrated herein for vibrational infusion into the skin surface 100. The vibration or motion of the paddle 60 of the present invention is represented by the arrow 104, and arrows 106 and 108 illustrate the infusion angles imparted by the vibrational action of the paddle 60 of the present invention in conjunction with the pattern 64 formed upon the face 62 thereof. In patients where it is determined that dermatological abrasion is not appropriate, the non-abrasive paddle 60 may be used.

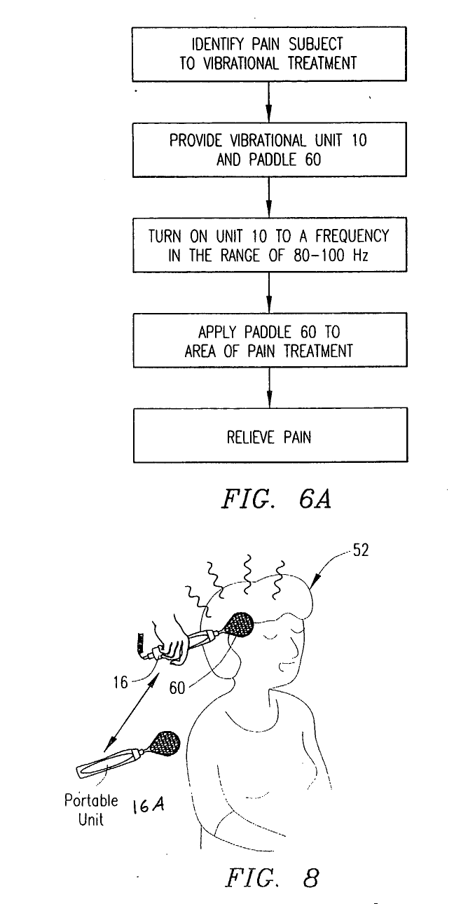

Referring now to FIG. 6, there is illustrated a flow diagram of one embodiment of the dermatological aspect of the present invention wherein step 198 comprises making the paddle 60 of the present invention and step 200 comprises the abrasive treatment of an area of the skin to expose an underlying layer of skin cells. Step 202 illustrates the application of a substance such as cream, ointment, lotion or the like, and/or other materials having dermatological ingredients adapted for skin rejuvenation. The application may be upon the treated skin area, upon the paddle 60 of the present invention or both. Step 204 is the activation of the vibrational energy of the paddle 60, and step 206 is the placing of the paddle 60 of the present invention upon the treated skin. In Step 208 the lotion, ointment, cream or other substance is infused into the skin area. These steps have been found to provide the pain treatment discussed herein, in addition to the dermatological effects.

FIG. 6A illustrates the use of the paddle 60 for headache pain relief. It is again noted that a battery powered vibrational unit is utilized which is; diagrammatically represented by vibrational wand unit 16A in the drawing of FIG. 8.

FIG. 7 illustrates yet another embodiment of the present invention wherein a two-sided paddle is shown. One side includes a permanent abrasive surface while the other side includes the surface 62 with the above-described pattern 64 thereon. The two-sided paddle may be bowed as shown or planar. In this way, a single paddle can be constructed with permanent abrasive and substantially non-abrasive surfaces for removing excess skin cells and substance infusion, respectfully. One technique would be to fabricate the two-sided paddle from structurally reinforced polycarbonate, polypropylene and/or polyethylene to provide a surface 62 on the one side an ultrasonically welded abrasive etched, stainless steel surface on the other. The use of the non-abrasive side would be possible for headache relief where the patient's skin is too sensitive for abrasive treatment.

FIG. 8 illustrates the use of the vibrational unit described herein for the relief of headaches. A portable, battery operated vibrational wand unit 16A is shown adjacent a patient to illustrate that either type of unit may be used. When headache pain is intense and the patient is not able to reach a medical care facility, arrangements may be made for personal access to and use of such a portable unit.

Applicants have determined that the combined treatment of abrasion and kinetic infusion of select substances on an area of skin provides multiple advantages to enhance both the appearance, as well as the overall health of a given skin area. Applicants have moreover, determined by discovery that pain treatment may also be expected when vibrational energy in the ranges described herein are used.

The provisional applications referenced above include multiple marketing materials describing various aspect of embodiments of certain aspects of the paddle of the present invention relative to dermatological applications.

In operation, the dermatological system of the present invention is specifically adapted for a dermatological infusion treatment modality in accordance with the principles of the present invention.

As noted above, a special, substantially non-abrasive surface such as that shown in FIG. 4 is provided in accordance with the principles of the present invention. In one embodiment, the present invention incorporates the use of the abrasive portion of the dermatological paddle to impart infusion into the skin. In other words, it has been found by the inventors of the present invention, that both an abrasive as well as a substantially non-abrasive paddle surface may be utilized for the beneficial effects of substance infusion into the skin of a patient being treated in accordance with principles of the present invention. In that regard, the application of cream or lotion to the skin area and/or to the paddle may be used in conjunction with a substantially non-abrasive or an abrasive paddle.

Although various embodiments of the method and apparatus of the present invention have been illustrated in the accompanying Drawings and described in the foregoing Detailed Description, it will be understood that the invention is not limited to the embodiments disclosed, but is capable of numerous rearrangements, modifications and substitutions without departing from the spirit of the invention as set forth herein.

1. A method for dermatological rejuvenation of a skin area of an individual utilizing vibratory motion of select dermatological treatment surfaces applied to the individual's skin, the method comprising the steps of:
providing a first dermatological treatment surface having a sufficiently abrasive side for removal of at least one outer skin area of an individual;
removing said at least one outer skin area of said individual through abrasive vibratory treatment of at least one said skin area with said first paddle and exposing an underlying skin area;
applying a cream, lotion, or other dermatological aid to said exposed underlying skin area of said individual;
providing a second dermatological treatment surface having a substantially non-abrasive side containing a plurality of lands and grooves formed therein; and
infusing said cream, lotion, or other dermatological aid into said exposed underlying skin area of said individual through vibratory movement of said second surface at a frequency less than on the order of 1000 Hz.

2. The method of claim 1 wherein said step of infusing includes the step of positioning the lands and grooves of said second dermatological treatment surface over said skin area to impart, by said vibratory movement, angular vectors to molecules of said cream, lotion, or other dermatological aid to drive said molecules into said exposed underlying skin area.

3. The method of claim 1 wherein said step of providing a first dermatological treatment surface further includes the step of providing a first paddle adapted to include said first dermatological treatment surface upon one of said first paddle's sides.

4. The method of claim 1 wherein said step of providing a second dermatological treatment surface further includes the step of providing a second paddle adapted to include said second dermatological treatment surface upon one of said second paddle's sides.

5. The method of claim 4 wherein said step of providing a second a paddle further includes the step of providing a paddle that is substantially round in shape.

6. The method of claim 4 wherein said step of providing a second a paddle further includes the step of providing a paddle that is substantially rectangular in shape.

7. The method of claim 1 wherein said step of infusing utilizes vibratory movement of said second dermatological treatment surface to impart vibratory movement generally on the order of 100 Hz.

8. The method of claim 5 wherein said step of infusing utilizes vibratory movement of said second dermatological treatment surface includes imparting vibratory movement generally on the order of the range of 80-100 Hz.

9. The method of claim 1 wherein said step of providing a second dermatological treatment surface includes the step of forming said second dermatological treatment surface with a plurality of lands of a regular shape defined by a pattern of recessed grooves, said lands being specifically adapted for vibratory infusion of said cream, lotion, or other dermatological aid.

10. The method of claim 1 wherein said step of providing a second dermatological treatment surface includes the step of forming said second dermatological treatment surface with a plurality of lands of an irregular shape defined by a multitude of recessed grooves, said lands being specifically adapted for vibratory infusion of said cream, lotion, or other dermatological aid.

11. The method of claim 1 wherein said step of providing a second dermatological treatment surface includes the step of forming said second dermatological treatment surface with a generally flat surface on which said plurality of lands resides.

12. The method of claim 1 wherein said step of providing a second dermatological treatment surface includes the step of forming said second dermatological treatment surface with a generally curved surface on which said plurality of lands resides.

13. A system for dermatological rejuvenation of an individual's skin area, the system comprising:
a first dermatological treatment surface having a sufficiently abrasive side for vibratory removal of an outer skin area of an individual;
a second dermatological treatment surface having a side specifically adapted for the vibratory infusion of a cream, lotion, or other dermatological aid into an underlying skin are of said individual;
a wand adapted to transfer a vibrational energy to said first and second dermatological treatment surfaces; and
a control unit connected to said wand and further capable of generating a vibrational energy in said wand at a frequency of generally less than 1000 Hz.

14. The system of claim 13 wherein said control unit is capable of producing an adjustable frequency generally within the range of 0 and 1000 Hz.

15. The system of claim 13 wherein said second dermatological treatment surface contains a plurality of lands of a regular shape defined by a pattern of recessed grooves, said lands being specifically adapted for vibratory infusion of said cream, lotion, or other dermatological aid.

16. The system of claim 13 wherein said second dermatological treatment surface contains a plurality of lands of an irregular shape defined by a plurality of recessed grooves, said lands being specifically adapted for vibratory infusion of said cream, lotion, or other dermatological aid.

17. The system of claim 13 wherein said second dermatological treatment surface has a substantially flat side on which the plurality of lands resides.

18. The system of claim 13 wherein said second dermatological treatment surface has a substantially curved surface on which the plurality of lands resides.

19. The system of claim 13 wherein said control unit is adapted to generate a vibrational energy in said wand on the order of 100 Hz.

20. The system of claim 13 wherein said control unit is adapted to generate a vibrational energy in said wand on the order of the range of 80-100 Hz.

21-36. (canceled)
